(19) United States
(12) Patent Application Publication
(10) Pub. No.: US 2002/0012960 A1
(43) Pub. Date: Jan. 31, 2002

(54) Method of finding protoporphyrinogen oxidase inhibitors

(76) Inventors: Otto Schallner, Monheim (DE); Werner Zitzmann, Leverkusen (DE); Klaus-Gunther Tietjen, Langenfeld (DE)

Correspondence Address: BAYER CORPORATION, PATENT DEPARTMENT, 100 BAYER ROAD, PITTSBURGH, PA, 15205, US

(21) Appl. No.: 09/897,233

(22) Filed: Jul. 2, 2001

(30) Foreign Application Priority Data
Jul. 5, 2000 (DE) 10032633.1

(51) Int. Cl. C12Q 1/26

(52) U.S. Cl. 435/25

(57) ABSTRACT

The present invention relates to a method of finding substances which interact with the enzyme protoporphyrinogen oxidase (PPO) and to a method for assaying the interaction of a substance with PPO. Furthermore, the invention relates to reagents for these methods, to a suitable assay system and to PPO inhibitors which are identified by the methods according to the invention.

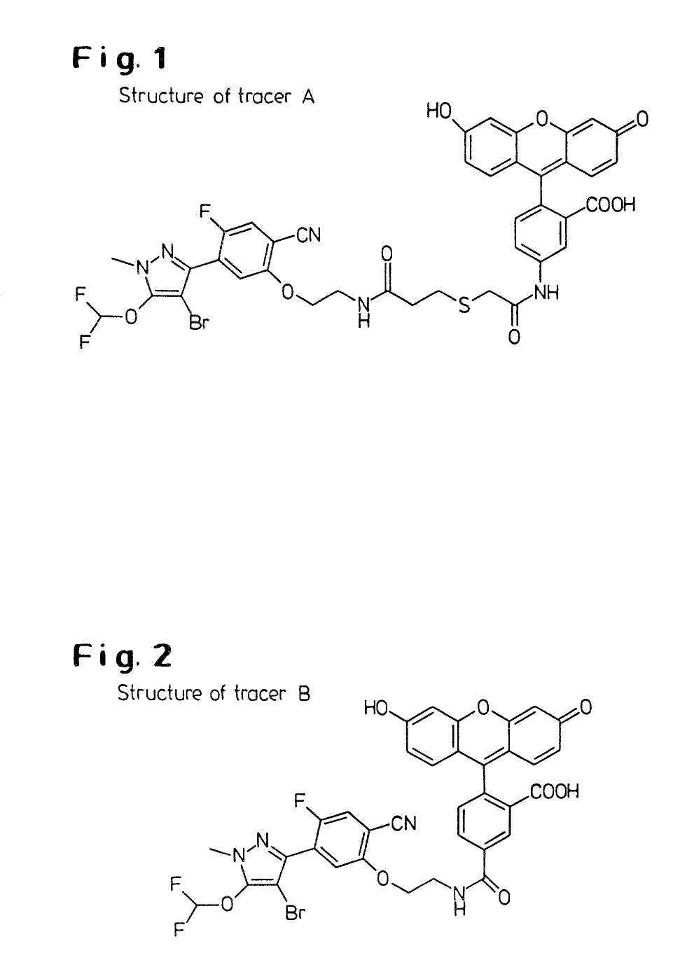

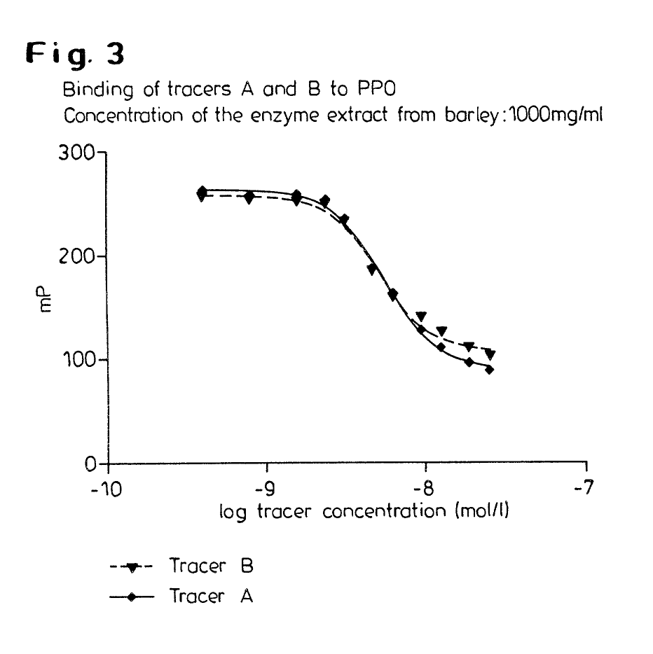

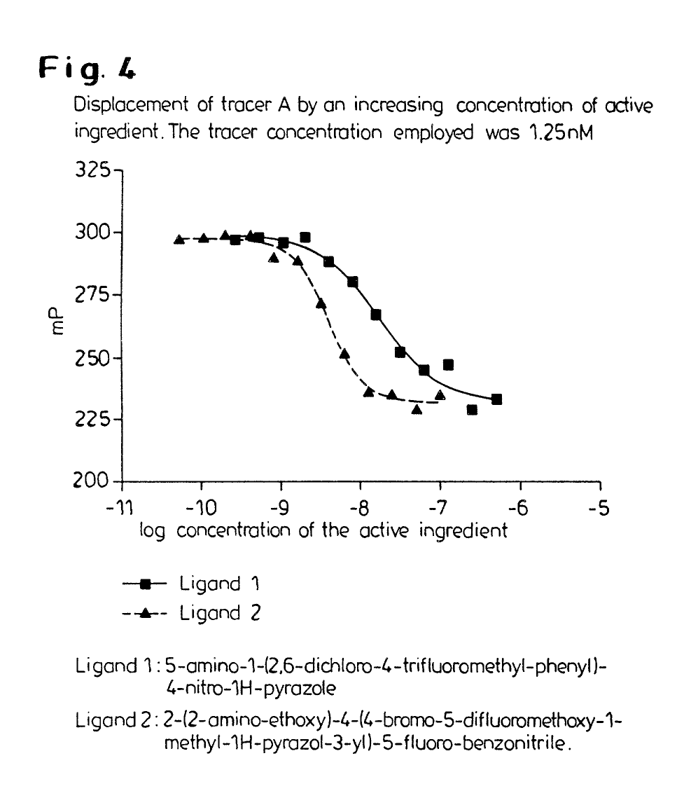

The present invention relates to a method of finding substances which interact with the enzyme protoporphyrinogen oxidase (PPO) and to a method for assaying the interaction of a substance with PPO. Furthermore, the invention relates to reagents for these methods, to a suitable assay system and to PPO inhibitors which are identified by the methods according to the invention.

Undesired plant growth can be prevented by using herbicides. The demands made to herbicides have been rising constantly with regard to their efficacy, costs and environmental compatibility. There exists therefore a demand for new substances which can be developed into potent new herbicides.

As a rule, herbicides have certain targets in the metabolism of the plants and act in many cases by inhibiting certain essential metabolic pathways of the plants. In many cases, herbicidal active compounds inhibit or block enzymes which make possible in the first place certain part-reactions of such metabolic pathways as biocatalysts. The plant enzymes or, in a more general formulation, proteins which constitute the biochemical basis for the action of herbicides or of substances with a potentially herbicidal action are termed target proteins.

A particularly important target for substances with herbicidal or growth-regulatory action is constituted by the enzymes of the biosynthetic pathway for porphyrines. This applies in particular to the enzyme protoporphyrinogen oxidase (PPO), which exists in two cellular forms. One of these two forms is located in the chloroplasts of the plants, while the other form is found in the mitochondria.

Within the biosynthetic pathway of the prophyrines, the enzyme PPO catalyses the oxidation of protoporphyrinogen IX to protoporphyrine IX (Matringe et al. 1989, Biochem. J. 260, 231). Porphyrines are of essential importance in a multiplicity of biochemical reactions which are important for the plant. These include, inter alia, the biosynthesis of the chlorophylls which play a central role in connection with the photosynthesis of the plants, and the biosynthesis of haeme, which is involved in further biologically important processes, such as, for example, cell restoration. Haeme is a cofactor of haemoglobin, cytochromes, p450-oxygenases, peroxidases and catalases.

The mechanism of action of herbicides which have PPO as their site of action is probably based on phototoxic reaction. Upon inhibition of the enzyme, its substrate protoporphyrinogen IX accummulates in the chloroplasts. It is possible that this compound leaks into the cytosol under the abovementioned conditions, where it is oxidized by a peroxidase to give protoporphyrine IX. Upon exposure to light, protoporphyrine IX can lead to the formation of singulet oxygen and further reactive oxygen species. As a consequence of the peroxidation of lipids, and membrane damage which this entails, the plant cells rapidly die (Lee et al. 1993, Plant Physiol. 102, 881).

Herbicidally active inhibitors of the enzyme PPO can be assigned to a series of different classes of substances, such as, for example, diphenyl ethers, oxadiazoles, cyclic imides, phenylpyrazoles and pyridine derivatives (Duke et al. 1991, Weed Sci. 39, 465; Nandihalli et al. 1992, Pesticide Biochem. Physiol. 43, 193; Yanase and Andoh 1989, Pesticide Biochem. Physiol. 35, 70).

Herbicidally active PPO inhibitors can be found for example by testing substances directly on plants. These tests are frequently carried out under greenhouse conditions. Suitably optimized methods make it possible to study a multiplicity of substances under those conditions for their biological action. Whether herbicidally active compounds which have been found in this manner indeed use PPO as target can, in the end, only be detected by enzymatic studies using isolated PPO enzyme.

To increase the throughput of substances to be tested, tests were developed which use miniaturized plant systems or parts of plants and plant cell cultures, and more and more such tests were carried out. However, when such miniaturized test systems are used, it may be difficult occasionally to achieve a sufficiently high sample throughput with readily reproducible test results.

An alternative of the abovementioned methods is the screening with biochemical assay methods. Typical biochemical methods comprise, as a rule, an isolation step and, if appropriate, subsequent purification of the enzyme from suitable material, in the case of herbicide targets plant material. Another possibility consists in making available the enzyme by cloning methods. In this case, the gene encoding the target is identified and expressed in a suitable heterologous system. Suitable expression systems are, for example, bacteria, yeasts, mammalian cells, insect cells and plants. Thereupon, the target protein can be purified and employed in specific assay methods.

A biochemical activity assay of the enzyme is generally carried out independently of whether the enzyme is obtained directly from plants or plant cell cultures or comparable systems, or else produced by recombinant methods in the broadest sense. In a conventional enzyme assay, the formation of a product is quantified with the aid of suitable measuring methods during the enzymatically catalysed reaction as a function of time. As an alternative, the decrease of a starting material during the course of the enzymatic reaction is determined in some cases. In such methods, the parameter consists, as a rule, in a measurable change in the absorption or fluorescence of the products or starting materials of such a reaction. In many cases, the reaction is also monitored with radiochemical methods. Instead of measuring the enzymatic activity directly, a biochemical screening for new active compounds may also be carried out with the aid of binding tests. In this case, detection of the inhibitors is not based on determining the inhibition of an enzymatic reaction as in the case of the abovementioned approaches, but on an analysis of the binding behaviour of test substances. The basis of this approach is the empirically concerned fact that inhibitors of enzymatic reactions must bind to the enzyme in most cases in order to be able to inhibit the enzyme in question.

A multiplicity of methods has been developed in the meantime for analyzing the binding of substances to enzymes and other protein molecules, such as, for example, receptors (see, for example, Hulme 1990, Receptor Biochemistry, IRL Press). In principle, one must differentiate between methods in which labelled compounds are employed and methods where such a label is not necessary. A disadvantage of such methods is, however, the fact that these methods are frequently not homogeneous. In many cases, the free ligand and the ligand bound to the target protein must be separated physically unless direct detection in the homogeneous system is possible.

It was therefore an object of the present invention to develop a method in which substances can be found which interact with PPO and thus constitute potential herbicidal active compounds while providing as high as possible a throughput of test compounds.

The object was achieved by providing a method for finding substances which interact with the enzyme PPO comprising the following steps:

a) preparing mixtures which comprise, in various concentrations, (i) PPO, (ii) a substance which is capable of interacting with PPO and which fluoresces when exposed to suitable irradiation, and (iii) a substance to be tested, or a mixture of substances to be tested,

b) irradiating the mixtures with plane-polarized light of a suitable wavelength which excites the fluorescent substance to emit light, and

c) measuring the fluorescence polarization values or the anisotropy values of the light emitted.

The method is based on measuring the change in fluorescence polarization in an assay system which takes place when a low-molecular-weight fluorescent substance (tracer) is displaced from PPO by a substance to be tested. The high sensitivity of fluorescence polarization assays makes it possible to detect even low concentrations of substances which interact with PPO. A decrease in the fluorescence polarization value with an increasing concentration of the substance to be tested, or the mixture of substances to be tested, indicates an interaction of one or more substances to be tested with PPO. The anisotropy of the measurement system may also be analyzed, and used for evaluating the experiments, in place of fluorescence polarization. In this context, fluorescence polarization and anisotropy are functionally equivalent methods (cf. J. R. Lakowicz, Principles of Fluorescence Spectroscopy, 2^{nd} Edition, Kluver Academic/Plenum Publishers, 1999).

The phenomenon of fluorescence polarization has already been known since Perrin (1926), who discovered that the fluorescent light emitted after exciting a fluorescent molecule with polarized light is also polarized in a certain plane if this molecule is stationary between excitation and emission (see F. Perrin 1926: “Polarisation de la lumiere de fluorescence. Vie moyenne des molecules dans l'état excité” [Polarization of fluorescent light. Half-life of molecules in the excited state], pages 390-401). In total, fluorescence polarization (or, as mentioned above, anisotropy) has been used very little indeed until recently in the sector of screening for new active compounds (see in this context the paper by J. R. Sportsman et al. 1997: “Fluorescence Polarization” in High Throughput Screening, Editor J. P. Devlin, Verlag Marcel Dekker). The situation in the field of medical diagnostics was, and is, quite different (see, in this context, I. Hemmilä 1991: “Applications of Fluorescence in Immuno-assays”, Wiley Interscience). The apparatuses employed in the field of diagnostics or in clinical chemistry are frequently cell-based apparatuses which are generally little suited for the high throughput of samples as is necessary in the field of active compound screening. As efficient and sensitive plate readers became available, the situation has changed so that a broad range of applications in biochemistry and molecular biology (see Checovich W., Bolger R. and Burke T. 1995, Nature 375, 254-256) has now become possible. Such plate readers for microtiter plates which are suitable for measuring the fluorescence polarization, or anisotropy, are now also used for screening active compounds, for example in the field of finding new pharmaceutical active compounds (see the above paper by J. R. Sportsman).

It was a further object of the present invention to develop a method for assaying whether a substance interacts with the enzyme PPO or not.

This object was achieved by providing a method for assaying whether a substance interacts with the enzyme protoporphyrinogen oxidase (PPO) comprising the following steps:

a) preparing mixtures which comprise, in various concentrations, (i) PPO and (ii) a substance which fluoresces when exposed to suitable irradiation,

b) irradiating the mixtures with plane-polarized light of a suitable wavelength which excites the fluorescent substance to emit light, and

c) measuring the fluorescence polarization values or the anisotropy values of the light emitted.

Thus, if it is suspected that a particular substance is capable of interacting with PPO, such an interaction can be detected by the method according to the invention by labelling the substance to be tested with a suitable fluorescent dye—unless the substance to be tested already has fluorescent properties per se—and bringing various concentrations of the resulting conjugate (termed tracer in the examples section) into contact with PPO. An increase in the fluorescence polarization value with a decreasing concentration of the fluorescent substance indicates an interaction with PPO since in accordance with a decreasing concentration of the fluorescent substance which is capable of binding to the target protein (of the tracer) increasingly large quantities of this compound are present in a form in which they are bound to the PPO molecule. Since free fluorescent substance (tracer) which is not bound to PPO is, accordingly, present in smaller quantities, the fluorescence polarization or anisotropy values measured rise.

Plant PPO is preferably employed in the methods according to the invention.

The plant PPO is especially preferably derived from barley or oats, very especially preferably from barley etioplasts. Obtaining the enzyme material from etioplasts has the advantage that relatively little background of compounds which might interfere with the measurement, for example chlorophylls, is present in the extract under these conditions.

Plant cell extracts which contain PPO can be used for the methods according to the invention. Such plant extracts are prepared by customary standard methods. As a rule, the plant material is homogenized, for which purpose various methods can be employed, such as, for example, mechanical homogenization, sonication methods and other disruption methods which are suitable for plant material (cf. in this context Deutscher 1990, Guide to Protein Purification, Methods in Enzymology 182).

However, PPO may also be isolated or purified biochemically from plant cell extracts. A particularly suitable enzyme material can be prepared by suitable methods. One example is the use of dialysis, which makes it possible to reduce the amount of compounds which can interfere with, or adversely affect, the measurements carried out in the methods according to the invention. This also includes reducing the viscosity of the enzyme preparation and thus also of the measurement batch, such as by reducing the sucrose content of the enzyme preparation.

Further suitable methods such as various chromatographic purification methods and precipitation and extractive methods which can lead to an increased PPO content or to a reduced content of components of the enzyme extract which interfere with the polarization measurement or its selectivity are found in current publications in the field of protein biochemistry (cf., for example, Deutscher 1990, Guide to Protein Purification, Methods in Enzymology 182; Marshak et al. 1996, Strategies for Protein Purification and Characterization, Cold Spring Harbor Lab., New York).

A further possibility consists in expressing the PPO-encoding gene in heterologous host cells and employing PPO in the form of a cell extract or in purified form. Suitable DNA which encodes plant PPO is described in WO 95/34659. The choice of suitable promotors for expressing the PPO-encoding DNA is within the knowledge of the skilled worker and depends on whether pro- or eukaryotic cells or cell-free systems are used for expression. Examples of heterologous promotors are the SV40, adenovirus or cytomegalovirus early or late promotors, the lac system, the trp system, the main operator and promotor regions of phage lambda, the control regions of the fd protein, the 3-phosphoglycerate kinase promotor, the acid phosphatase promotor and the yeast α-mating factor promotor. Vectors which may be used are all plasmids, phasmids, cosmids, YACs or artificial chromosomes which are used in molecular biology laboratories. Suitable host cells are not only prokaryotic cells such as the bacteria from the genera Bacillus, Pseudomonas, Streptomyces, Streptococcus, Staphylococcus, preferably E. coli, but also eukaryotic cells such as yeasts, mammalian cells, insect cells or plant cells. Preferred eukaryotic host cells are, inter alia, insect cell lines such as Sf9 which can be employed as host cells within the baculovirus expression systems.

A rapid method of isolating PPO which has been synthesized by host cells starts with the expression of a fusion protein, it being possible for the fusion partner to be affinity-purified in a simple manner. For example, the fusion partner may be glutathione S-transferase. The fusion protein can then be purified on a glutathione affinity column. If required, the fusion partner can be separated by partial proteolytic cleavage, for example at linkers between the fusion partner and the polypeptide according to the invention to be purified. The linker can be designed such that it includes target amino acids, such as arginine and lysine residues, which define sites for trypsin cleavage. Standard cloning methods using oligonucleotides may be employed for generating such linkers.

Further modifications can be particularly beneficial for the concentration and purification of cloned proteins, for example cloning and expressing PPO which is provided with His tags. This makes isolation with suitable affinity methods particularly simple (cf. company brochures for example of the companies Novagen, Qiagen and Amersham Pharmacia; on cloning and expression methods in general: Sambrook et al. 1989, Molecular Cloning, A Laboratory Handbook, 2^{nd} ed., Cold Spring Harbor Lab., New York).

Suitable purification methods are based on preparative electrophoresis, FPLC, HPLC (for example using gel filtration columns, reversed-phase columns or moderately hydrophobic columns, gel filtration, differential precipitation, ion-exchange chromatography and affinity chromatography, including nickel chelate chromatography and related methods. These methods can be employed for isolating and purifyng PPO from plant or animal cell material and from bacteria, yeasts or fungi.

The substance which fluoresces upon exposure to suitable irradiation which is capable of interacting with PPO and which is to be employed in the methods according to the invention is preferably a PPO ligand, a natural PPO substrate, a natural product of the PPO enzyme reaction or a herbicidally active PPO inhibitor.

The substances themselves may already have suitable fluorescent properties or may be labelled with a fluorescent dye.

The fluorescent substance or fluorescent dye with which a substance which is capable of interacting with PPO is labelled should preferably have an excitation maximum in the range of from 250 to 750 nm, an emission maximum in the range of from 300 to 900 nm and a rotational relaxation time in the range of from 1 to 10 nanoseconds. The fluorescent substance or the fluorescent dye should preferably have properties which lead to polarization values of between 0 and 400 mP. Examples of such dyes which are suitable for labelling are the dyes fluorescein, fluorescein derivative& such as fluorescein isothiocyanate, rhodamine, dansyl derivatives such as dansyl chloride, coumarins or coumarin derivatives such as umbelliferone, fluorophores from the group of the Alexa fluorescent dyes (ALEXA Dyes, Molecular Probes Inc., Eugene, USA), such as [1,10-dihydro-2,2,10,10-tetramethyl-4,8-bis(sulphomethyl)-2H-pyrano[3,2-g:5,6-g′]diquinolin-6-yl]-benzoic acid, 6-[2-carboxyphenyl]-1,2,10,11-tetrahydro-1,2,2,10,10,11-hexamethyl-4,8-bis(sulphomethyl)-pyrano[3,2-g:5,6-g′]di-quinolin-13-ium salt, 6-[2-carboxy-5-[(carboxymethyl)thio]-3,4,6-trichlorophenyl]-1,2,3,4,8,9,10,11-octahydro-2,2,4,8,10,10-hexamethyl-12,14-disulfo-pyrano[3,2-g:5,6-g′]diquinolin-13-ium salt, 5-(4-carboxyphenyl)-1,2,3,7,8,9-hexahydro-2,3,3,7,7,8-hexamethyl-10,12-disulpho-pyrano[3,2-f:5,6-f]diindol-11-ium salt, 3,6-diamino-9-(2-carboxy-phenyl)-4,5-disulpho-xanthylium salt, 8,8-dimethyl2-oxo-6-(sulphomethyl)-4-(trifluoromethyl)-2H-pyrano[3,2-g]quinoline-9(8H)-hexanoic acid, 7-amino-3-methylcarboxy-4-methyl-2-oxo-2H-1-benzopyran-6-sulphonic acid, 2-(2,7-dichloro-9,9a-dihydro-6-hydroxy-3-oxo-3H-xanthen-9-yl)-benzoic acid, 2-(2,7-difluoro-9,9a-dihydro-6-hydroxy-3-oxo-3H-xanthen-9-yl)-benzoic acid, 2-(2,4,5,7-tetrabromo-9,9a-dihydro-6-hydroxy-3-oxo-3H-xanthen-9-yl)-benzoic acid, 2-(2,7-dichloro-4-nitro-9,9a-dihydro-6-hydroxy-3-oxo-3H-xanthen-9-yl)-benzoic acid, 2-(9,9a-dihydro-6-hydroxy-3-oxo-2,4,5,7-tetraiodo-3H-xanthen-9-yl)-benzoic acid, 2-(4,5,6-trihydroxy-3-oxo-3H-xanthen-9-yl)-benzoic acid, and the respective derivatives thereof, fluorophores from the group of the CyDyes fluorescent dyes (Amersham Life Sciences, UK) and from the group of the Bodipy fluorescent dyes (Molecular Probes Inc., Eugene, USA).

Fluorescein is preferably used as fluorescent dye.

The fluorescent substance especially preferably has the following structure:

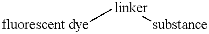

where

“linker” represents a hydrocarbon chain which is in each case straight-chain or branched, in each case saturated or unsaturated, in each case optionally substituted, in each case linked at one end to the substance and at the other end to the fluorescent dye, it being possible for this hydrocarbon chain to contain in each case at the beginning or at the end or within the chain one or more of the following hetero components:

Q1, CQ², CQ²-Q¹, Q¹-cQ², Q¹-CQ²-Q¹, SO, SO₂

where in each case Q¹ and Q² represent O, S or NH,

or represents a carbocyclic or heterocyclic group which is in each case saturated or unsaturated, in each case optionally substituted and linked at one end to the substance and at the other end to the fluorescent dye, and

“substance” represents a substance which is capable of interacting with PPO.

“Fluorescent dye” preferably represents a dye group which is characterized by the formula hereinbelow

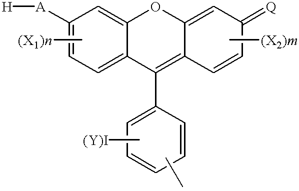

where

A represents O or NH,

Q represents O, S or NH,

X¹ represents hydrogen, halogen, nitro, hydroxyl, carboxyl (COOH), sulpho (SO₃H) or alkyl,

X² represents hydrogen, halogen, nitro, hydroxyl, carboxyl (COOH), sulpho (SO₃H) or alkyl,

Y represents halogen, alkyl, carboxyl (COOH) or sulpho (SO₃H),

l represents the indices 0 to 4, and

m, n represents the indices 0 to 3.

A preferably represents O.

Q preferably represents O.

X¹ preferably represents hydrogen, fluorine, chlorine, bromine or alkyl having 1to 4 carbon atoms.

X² preferably represents hydrogen, fluorine, chlorine, bromine or alkyl having 1 to 4 carbon atoms.

X¹ especially preferably represents hydrogen, fluorine, chlorine, methyl or ethyl.

X² preferably represents hydrogen, fluorine, chlorine, methyl ethyl.

The hydrocarbon chain of the linker preferably has up to 10 carbon atoms.

The carbocyclic or heterocyclic group of the linker preferably has up to 10 carbon atoms and, if appropriate, up to 5 nitrogen atoms and, if appropriate, 1 or 2 oxygen or sulphur atoms.

The substance preferably has the following structure:

Z¹—Al¹—Z—

where

A¹ represents a single bond, O or S,

Z¹ represents a carbocyclic or heterocyclic group having in each case up to 10 carbon atoms and, if appropriate, up to 5 nitrogen atoms and, if appropriate, 1 or 2 oxygen or sulphur atoms, and

Z² represents a carbocyclic or heterocyclic group having in each case up to 10 carbon atoms and, if appropriate, up to 5 nitrogen atoms and, if appropriate, 1 or 2 oxygen or sulphur atoms.

A¹ preferably represents a single bond.

Z¹ preferably represents one of the following groups

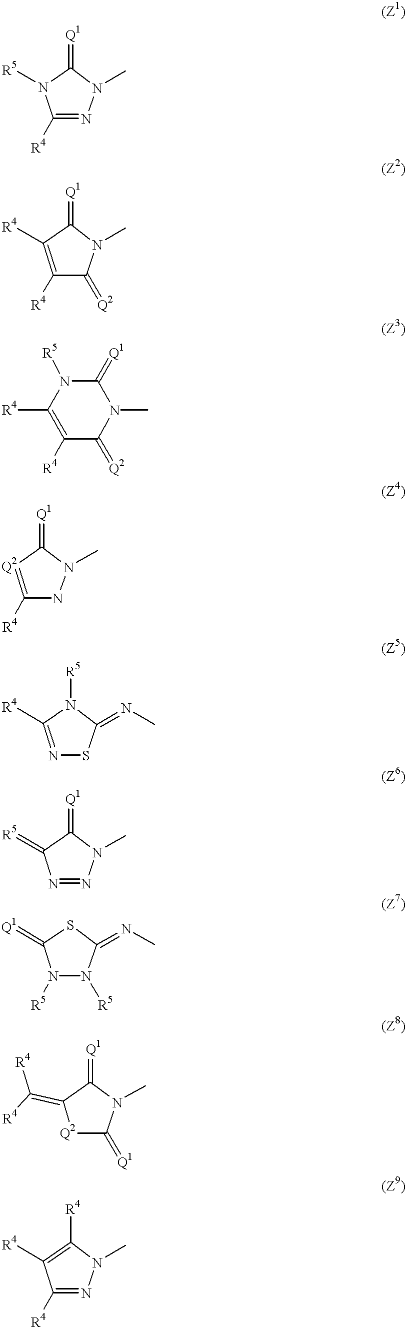

where

Q¹ represents O or S,

Q² represents O or S,

R⁴ represents hydrogen, amino, nitro, cyano, carboxyl, carbamoyl, thio-carbamoyl, halogen, alkyl which has 1 to 6 carbon atoms and which is optionally substituted by cyano, halogen or C₁-C₄-alkoxy, or represents alkenyl or alkinyl, each of which has 2 to 6 carbon atoms and each of which is optionally substituted by halogen or represents alkoxy or alkoxycarbonyl, each of which has 1 to 6 carbon atoms in the alkyl groups and each of which is optionally substituted by cyano, halogen or C₁-C₄-alkoxy, or represents alkenyloxy or alkinyloxy, each of which has 3 to 6 carbon atoms and each of which is optionally substituted by halogen, or represents alkylthio which has 1 to 6 carbon atoms which is optionally substituted by cyano, halogen or C₁-C₄-alkoxy, or represents alkenylthio or alkinylthio, each of which has 3 to 6 carbon atoms and each of which is optionally substituted by halogen, or represents alkylamino or dialkylamino, each of which has 1 to 6 carbon atoms in the alkyl groups, or represents cycloalkyl or cycloalkylalkyl, each of which has 3 to 6 carbon atoms in the cycloalkyl groups and, if appropriate, 1 to 4 carbon atoms in the alkyl moiety and each of which is optionally substituted by cyano, halogen or C₁-C₄-alkyl,

R⁵ represents hydrogen, hydroxyl, amino, cyano, or represents alkyl, alkoxy, alkoxycarbonyl or alkylamino, each of which has up to 6 carbon atoms and each of which is optionally substituted by cyano, halogen or C₁-C₄-alkoxy, or represents alkenyl or alkinyl, each of which has up to 6 carbon atoms and each of which is optionally substituted by halogen, or represents cycloalkyl or cycloalkylalkyl, each of which has 3 to 6 carbon atoms in the cycloalkyl groups and, if appropriate 1 to 4 carbon atoms in the alkyl moiety and each of which is optionally substituted by cyano, halogen or C₁-C₄-alkyl, or represents phenyl or phenyl-C₁-C₄-alkyl each of which is optionally substituted by nitro, cyano, halogen, C₁-C₄-alkyl, C₁-C₄-halogenoalkyl, C₁-C₄-alkoxy or C₁-C₄-halogenoalkoxy,

Y¹ represents O, S, SO, SO₂, NH, N(alkyl) or methylene and

y² represents a single bond or O, S, SO, SO₂, NH or N(alkyl),

Y¹ and Y² being different in each individual case.

Z² preferably represents the following group

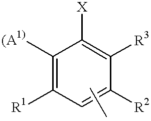

where

R¹ is hydrogen, nitro, cyano or halogen,

R² is nitro, hydroxyl, cyano, carbamoyl, thiocarbamoyl, or represents alkyl or alkoxy, each of which has 1 to 4 carbon atoms and each of which is optionally substituted by halogen, and

R³ represents nitro, hydroxyl, mercapto, amino, hydroxyamino, cyano, carboxyl, carbamoyl, thiocarbamoyl, halogen, or represents alkyl, alkylcarbonyl, alkoxy, alkoxycarbonyl, alkylthio, alkylsulphinyl, alkylsulphonyl or alkylamino, each of which has 1 to 6 carbon atoms in the alkyl groups and each of which is optionally substituted by cyano, carboxyl, carbamoyl, halogen, C₁-C₄-alkoxy or C₁-C₄-alkoxy-carbonyl, or represents alkylsulphonylamino, N,N-bis-alkylsulphonyl-amino, N-alkylcarbonyl-N-alkylsulphonyl-amino, each of which has 1 to 4 carbon atoms in the alkyl groups and each of which is optionally substituted by halogen, or represents alkenyl, alkenyloxy, alkenylthio, alkenylamino, alkinyl, alkinyloxy, alkinylthio, alkinylamino, each of which has up to 6 carbon atoms in the alkenyl or alkinyl groups and each of which is optionally substituted by cyano, carboxyl, carbamoyl, halogen or C₁-C₄-alkoxy-carbonyl, or represents cycloalkyl, cyclo-alkyloxy, cycloalkylthio, cycloalkylamino, cycloalkylsulphonylamino, cycloalkylalkyl, cycloalkylalkoxy, cycloalkylalkylthio or cycloalkylalkylamino, each of which has 3 to 6 carbon atoms in the cycloalkyl groups and, if appropriate, 1 to 4 carbon atoms in the alkyl moieties and each of which is optionally substituted by cyano, carboxyl, carbamoyl, halogen or C₁-C₄-alkyl, or represents aryl, aryloxy, arylthio, arylamino, arylalkyl, arylalkoxy, arylalkylthio, aryl-alkylamino, N-arylcarbonyl-N-alkylsulphonyl-amino, each of which has 6 or 10 carbon atoms in the aryl groups and, if appropriate, 1 to 4 carbon atoms in the alkyl moiety and each of which is optionally substituted by nitro, cyano, carboxyl, carbamoyl, halogen, C₁-C₄-alkyl, C₁-C₄-halogenoalkyl, C₁-C₄-alkoxy, C₁-C₄-halogenoalkoxy or C₁-C₄-alkoxy-carbonyl, and

X represents hydrogen or halogen.

A substance which is especially emphasized is furthermore one of the following structure:

Z¹—A¹—Z²—

where

A¹ represents O,

Z¹ represents one of the following groups

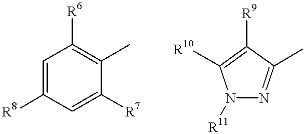

where

R⁶ represents cyano or halogen,

R⁷ represents hydrogen or halogen,

R⁸ represents cyano, halogen, or represents halogenoalkyl or halogeno-alkoxy, each of which has 1 to 4 carbon atoms,

R⁹ represents hydrogen or halogen,

R¹⁰ represents halogenoalkyl or halogenoalkoxy, each of which has 1 to 4 carbon atoms, and

R¹¹ represents alkyl having 1 to 4 carbon atoms, and

Z² represents the following group

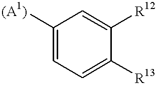

in which

R¹² represents carboxyl, or represents alkoxy or alkoxycarbonyl, each of which has up to 4 carbon atoms and each of which is optionally substituted by cyano, halogen or C₁-C₄-alkoxy, and

R¹³ represents cyano, carbamoyl, thiocarbamoyl, halogen or represents halogenoalkyl or halogenoalkoxy, each of which has 1 to 4 carbon atoms.

The substance especially preferably has the following general formula:

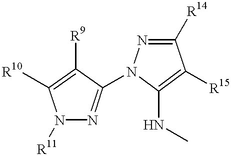

in which

R⁹, R¹⁰ and R¹¹ are as defined above,

R¹⁴ represents hydrogen, halogen or alkyl having 1 to 4 carbon atoms, and

R¹⁵ represents nitro, cyano, carbamoyl, thiocarbamoyl, or represents alkyl or alkoxy, each of which has 1 to 4 carbon atoms and each of which is optionally substituted by cyano, halogen or C₁-C₄-alkoxy.

Substances labelled with a fluorescent dye, as they are described hereinabove, are subject matter of the present invention per se.

Suitable substances to be tested which can be employed in the methods according to the invention are, for example, organochemical molecules, natural materials and compounds derived therefrom, or peptides. Substances which are preferably employed are small organochemical molecules or mixtures of these, which have been synthesized, for example, with the aid of combinatory chemistry. Constituents from culture media, cell extracts or conditioned media may also be employed.

The concentration of the fluorescent substance in the assay mix is preferably less than 1 μM, especially preferably less than 100 nM and very especially preferably less than 10 nM.

A multiplicity of various further reagents can be added to the assay mix. These include the various reagents such as, for example, salts, buffers, proteins such as, for example, albumin or gamma-globulins, detergents, protease inhibitors, solvents and the like. The purpose of such additives is either to influence the binding reaction directly, either by increasing the binding strength, by reducing the binding strength or by generating an advantageous modification (for the purposes of the assay) in the kinetics of the binding, to the target protein PPO, of the fluorescent substance or the substance to be tested or of the mixture of substances to be tested. Alternatively, the purpose of these additives may also be, for example, a stabilization of the target protein PPO, for example by inhibiting any proteases which may be present in the assay mix, by a positive effect on the solubility of the individual components in the assay mix, or by preventing any quenching effect in the assay mix which may occur.

The assay mix is incubated under conditions under which the fluorescent substance is capable of binding to the target enzyme PPO.

In one embodiment of the invention, all components of the mixture are in solution or in suspension when the assay mix contains insoluble components, for example sparingly soluble test substances, or when the target protein PPO is employed as crude extract.

The constituents of the mixture in the assay mix can be combined in any sequence as long as a suitable molecular interaction of the components of the mixture, in particular binding of the fluorescent substance to the target protein, is possible under these conditions.

Incubation is carried out under suitable temperatures. Incubation temperatures which are suitable are, for example, between 4° C. and 60° C., preferably between 15° C. and 45° C. and especially preferably between 20° C. and 40° C. The mix may be shaken during incubation.

The assay is carried out in various customary formats in microtiter plates or on surfaces or in cell or capillary systems.

Subject of the present invention is also an assay system comprising

a) containers with mixtures as defined above,

b) a device for irradiating the mixtures of plane-polarized light of a wavelength which excites the fluorescent substance to emit light, and

c) a device for measuring the fluorescence polarization values or the anisotropy values of the light emitted.

Subject-matter of the present invention are furthermore substances which interact with the enzyme protoporphyrinogen oxidase and which have been identified by the method according to the invention. Such substances constitute potential herbicides.

The substances according to the invention which are labelled with a fluorescent dye can also be employed in assay methods which are based on fluorescence correlation spectroscopy (cf. Auer et al. 1998, Fluorescence correlation spectroscopy: lead discovery by miniaturized HTS, Drug Discovery Today 3, 457).

The present invention is illustrated in greater detail hereinbelow with reference to examples.

### EXAMPLES

### Example 1

Synthesis of ligand 2: 2-(2-Amino-ethoxy)-4-(4-bromo-5-difluoromethoxy-1-methyl-1 H-pyrazol-3-yl)-5-fluoro-benzonitrile

0.31 g (7.7 mmol) of a 60% sodium hydride dispersion in liquid paraffin is added to a solution of 1.34 g (22 mmol) of ethanolamine in 30 ml of anhydrous acetonitrile. The mixture is stirred at room temperature for 30 minutes and a solution of 2.0 g (5.5 mmol) of 4-(4-bromo-5-difluoromethoxy-1-methyl-1H-pyrazol-3-yl)-2,5-di-fluoro-benzonitrile is then added. The reaction solution is warmed for six hours at 50° C., with stirring, then poured into 100 ml of ice-water and the emulsion formed is extracted with dichloromethane. The organic phase is washed in succession with water and saturated sodium chloride solution, dried over magnesium sulphate and freed from solvent in vacuo. The oily residue is stirred in succession with water and diethyl ether, and the solid is filtered off with suction and dried in vacuo at 40° C.

This gives 0.87 g (40% of theory) of 2-(2-amino-ethoxy)-4-(4-bromo-5-difluoro-methoxy-1-methyl-1H-pyrazol-3-yl)-5-fluoro-benzonitrile. m.p.: 101° C.

### Example 2

Conjugation of Ligand 2 with Fluorophoric Groups

Preparation of tracer A: conjugation of ligand 2 with fluorescein 5-EX, succinimidyl ester

Solution 1: 5.66 mg of ligand 2 (=2-(2-aminoethoxy)-4-(4-bromo-5-difluoro-methoxy-1-methyl-1H-pyrazol-3-yl)-5-fluoro-benzonitrile) are dissolved in 1.415 ml of DMSO.

Solution 2: 10 mg of fluorescein 5-EX,SE (Molecular Probes, Order No. F-6130) are dissolved in 0.25 ml of DMSO.

Solutions 1 and 2 are combined and incubated for 1 hour in the dark at room temperature.

The reaction product is subsequently purified by semipreparative HPLC using the following instrument parameters:

HPLC apparatus: Äkta Explorer (Amersham Pharmacia)

Stationary phase: NUCLEOSIL 100-7 C 18 by Macherey & Nagel (7 μm; 250×10 mm; column volume approx. 19.63 ml). This column was employed for the semipreparative purification of the product (tracer A). 250 μl of sample were applied and fractionated per run.

Elution: The column was eluted with a gradient of eluent A (0.5% formic acid, analytical grade) and eluent B (acetonitrile); the flow rate was 6 mllmin.

The gradient was carried out as follows:

Start: 50% A/50% B; in 5.5 column volumes to 35% A/65% B.

Detection: The reaction product (tracer A) was detected by UV measurement at 495 nm. The retention time was 9.0 min.

The selected fractions were combined, evaporated on a rotary evaporator in vacuo at 22° C. and freeze-dried. The structures were assigned with the aid of mass spectrometry.

In total, 2.8 mg of the product termed tracer A were obtained.

Molar mass =878; empirical formula: C₃₉H₂₉BrF₃N₅O₉S

### Example 3

Preparation of tracer B: Conjugation of ligand 2 with 5-carboxyfluorescein, succinimidyl ester

Solution 1: 1 mg of ligand 2 (2-(2-aminoethoxy)-4-(4-bromo-5-difluoromethoxy-1-methyl-1H-pyrazol-3-yl)-5-fluoro-benzonitrile) was dissolved in 1 ml of PBS/DMSO (60: 40).

Preparation of PBS (phospate-buffered saline): 8 g of NaCl, 0.2 g of KCl, 144 g of Na₂HPO₄ and 0.24 g of KH₂PO₄ were dissolved in 800 ml of distilled water, the pH was brought to 7.4, and the mixture was made up to 1 liter with distilled water.

Solution 2: 4 mg of 5-FAM,SE (5-carboxyfluorescein, succinimidyl ester, single isomer, Molecular Probes, Order No. C-2210) were dissolved in 0.4 ml of acetonitrile/DMSO (1:1). For the labelling mix, 0.2 ml of solution 2 was added to 1 ml of solution 1 and the mixture was incubated for 5 hours in the dark at room temperature.

The product was separated and tracer B was purified under the same conditions as in Example 2 with the aid of HPLC using an Akta HPLC system. The retention time of tracer B was 10.5 min.

The yield of product (tracer B) was 1.1 mg. Analysis by means of mass spectrometry revealed a molar mass of 763 g/mol.

Empirical formula of tracer B: C₃₅H₂₂BrF₃N₄O₈

### Example 4

Preparation of a PPO-containing Crude Protein Extract from Barley

Barley was sown in vermiculite and grown for 6 days in the dark at room temperature, in each case with exposure to light for 2 hours.

5 g of leaves from the barley plants obtained were comminuted and homogenized at 4° C. together with 20 ml of processing buffer.

The homogenate was filtered through nylon gaze (mesh size 0.1 mm). The plastid fraction was obtained from the filtrate by centrifugation; centrifugation was carried out at 4° C. in a tabletop centrifuge at 2500 revolutions per minute (Eppendorf, 5810 R, Rotor type A-4-62). The centrifugation time was 10 minutes. After centrifugation, the supernatant was decanted off and the pellet was suspended in 1 ml of processing buffer. The processing buffer was composed as follows: 350 mM sucrose, 10 mM KH₂PO₄, 10 mM Tris.HCl, 1 mM EDTA, 1 mM MgCl₂, 10 mM 2-mercaptoethanol, 0.1% BSA, brought to pH 7.4 with HCl and NaOH.

The PPO-containing barley etioplast fraction thus prepared had a protein content of 2.2 mg/ml. The protein content was determined with the aid of the Bio-Rad protein assay (Bradford, 1976; cf. Instructions by Bio-Rad), it was carried out as a micro-assay. 0.8 ml of a dilution series of the protein extract were treated in each case with 0.2 ml of Dye Reagent Concentrate and Vortex. After 10 minutes, the optical density was measured at 595 nm in a photometer (Beckmann spectrophotometer type DU 640) and the relevant value was read off from a protein standard curve; bovine serum albumin (BSA) was used as the protein standard.

### Example 5

Binding of Tracers A and B to the Target Enzyme PPO

To detect the binding to the target protein PPO, tracer A and tracer B were dissolved in DMSO; the concentration of this stock solution was 1 μM. The following were pipetted into the wells of a black 96-well microtiter plate (Greiner; FIA plates F-form, black):

1) 2.5 μl of a dilution of the tracer stock solution in question; depending on the mix, the end concentration of the tracer in question was between 5×10⁻⁷ and 5×10⁻¹⁰ mol/l;

2) 97.5 μl of barley PPO solution (protein end concentration: 1000 μg/ml in the mix).

The mix was subsequently incubated in an Eppendorf thermomixer for 10 minutes at room temperature (approx. 22° C.) with shaking (at a shaking motion of 650 revolutions per minute). The DMSO content in the assay mix was 2.5%.

The polarization of the mix was then measured in an Ultra plate reader (Tecan). Alternatively, the anisotropy of the mixture may be determined.

The conditions for measuring the polarization were as follows: Excitation wavelength (λₑₓ): 485 nm

Emission wavelength (λₑₘ): 530 nm

Number of light flashes per well: 30

The gain of the apparatus was set manually at 60.

Standards by PanVera (High and Low Polarization Standards) were used as reference.

### Example 6

Binding of Potentially Herbicidally Active Compounds to PPO: Assay of the Substances 5-amino-1-(2,6-dichloro-4-trifluoromethyl-phenyl)-4-nitro-1H-pyrazole and 2-(2-aminoethoxy)-4-(4-bromo-5-difluoromethoxy-1-methyl-1H-pyrazol-3-yl)-5-fluoro-benzonitrile

The protein solution (cf. Example 5, enzyme extract from barley; protein concentration 2.2 mg/ml) was diluted with potassium phosphate buffer pH 8.0 to a protein concentration of 1 mg/ml.

The following were pipetted into the wells of a black 96-well microtiter plate (Greiner; FIA plates F-form black):

1) 10.5 μl of the tracer solution (250 nM tracer A in DMSO); the end concentration of tracer was 1.25 nM;

2) 2.5 μl of a solution of the test substances in DMSO;

3) 97 ml PPO solution from barley.

The mix was subsequently incubated in an Eppendorf thermomixer for 10 minutes at room temperature (approx. 22° C.) with shaking (at a shaking motion of 650 revolutions per minute). The DMSO content in the assay mix was 3%.

The polarization of the mix was then measured in an Ultra plate reader (Tecan). Alternatively, the anisotropy of the mixture was determined. The conditions for measuring the polarization were as follows:

Excitation wavelength (λₑₓ): 485 nm

Emission wavelength (λₑₘ): 535 nm

Number of light flashes per well: 30

The gain of the apparatus was set manually at 75.

Standards by PanVera (High and Low Polarization Standards) were used as reference. As is customary for polarization measurements, the unit mP was used to state the polarization values.

The experiment was evaluated with the aid of the Software Graph Pad Prism (GraphPadSoftware, Inc.).

The following IC₅₀ values were obtained:

Ligand 1 (=5-amino-1-[2,6-dichloro-4-(trifluoromethyl)phenyl]-4-nitro-1H-pyrazole): IC₅₀=1.72×10^{31 8} mol/l

Ligand 2 (=2(2-aminoethoxy)-4[4-bromo-5-(difluoromethoxy)-1-methyl-1H-pyrazol-3-yl]-5-fluoro-benzonitrile): IC₅₀=3.94×10⁻⁹ mol/l.

1. Method for finding substances which interact with the enzyme protoporphyrinogen oxidase (PPO) comprising the following steps:
a) preparing mixtures which comprise, in various concentrations, (i) PPO, (ii) a substance which is capable of interacting with PPO and which fluoresces when exposed to suitable irradiation, and (iii) a substance to be tested, or a mixture of substances to be tested,
b) irradiating the mixtures with plane-polarized light of a suitable wavelength which excites the fluorescent substance to emit light, and
c) measuring the fluorescence polarization values or the anisotropy values of the light emitted,
where a decrease in the fluorescence polarization value with an increasing concentration of the substance to be tested or of the mixture of substances to be tested indicates an interaction of one or more substances to be tested with PPO.

2. Method for assaying whether a substance interacts with the enzyme protoporphyrinogen oxidase (PPO) comprising the following steps:
a) preparing mixtures which comprise, in various concentrations, (i) PPO and (ii) a substance which fluoresces when exposed to suitable irradiation,
b) irradiating the mixtures with plane-polarized light of a suitable wavelength which excites the fluorescent substance to emit light,
c) measuring the fluorescence polarization values or the anisotropy values of the light emitted,
where an increase in the fluorescence polarization value with a decreasing concentration of the fluorescent substance indicates an interaction with PPO.

3. Method according to claim 1 or 2, characterized in that the PPO is a plant PPO.

4. Method according to one of claims 1 to 3, characterized in that a PPO-containing plant cell extract is employed.

5. Method according to one of claims 1 to 3, characterized in that biochemically purified PPO from plant cell extracts is employed.

6. Method according to one of claims 1 to 3, characterized in that recombinantly produced PPO is employed.

7. Method according to one of claims 1 to 6, characterized in that the fluorescent substance is a substance labelled with a fluorescent dye.

8. Method according to claim 7, characterized in that the fluorescent dye is fluorescein or a fluorescein derivative.

9. Method according to one of claims 1 to 8, characterized in that the substance which is capable of interacting with PPO is a PPO ligand, a natural PPO substrate, a natural product of the PPO enzyme reaction or a herbicidally active PPO inhibitor.

10. Method according to one of claims 1 to 9, characterized in that the fluorescent substance has the following structure: 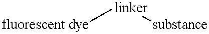
where
“linker”
represents a hydrocarbon chain which is in each case straight-chain or branched, in each case saturated or unsaturated, in each case optionally substituted, in each case linked at one end to the substance and at the other end to the fluorescent dye, it being possible for this hydrocarbon chain to contain in each case at the beginning or at the end or within the chain one or more of the following hetero components:
Q¹, CQ², CQ²-Q¹, Q¹-CQ², Q¹-CQ²-Q¹, SO, S₂
where in each case Q¹ and Q² represent O, S or NH,
or represents a carbocyclic or heterocyclic group which is in each case saturated or unsaturated, in each case optionally substituted and linked at one end to the substance and at the other end to the fluorescent dye, and
“substance”
represents a substance which is capable of interacting with PPO.

11. Substance labelled with a fluorescent dye, characterized by the following structure: 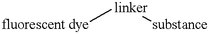
where
“linker”
represents a hydrocarbon chain which is in each case straight-chain or branched, in each case saturated or unsaturated, in each case optionally substituted, in each case linked at one end to the substance and at the other end to the fluorescent dye, it being possible for this hydrocarbon chain to contain in each case at the beginning or at the end or within the chain one or more of the following hetero components:
Q¹, CQ², CQ²-Q¹, Q¹-CQ², Q¹-CQ²-Q¹, SO, SO₂
where in each case Q¹ and Q² represent O, S or NH,
or represents a carbocyclic or heterocyclic group which is in each case saturated or unsaturated, in each case optionally substituted and linked at one end to the substance and at the other end to the fluorescent dye, and
“substance”
represents a substance which is capable of interacting with PPO.

12. Substance according to claim 11, characterized in that “fluorescent dye”
represents a dye group which is characterized by the formula hereinbelow 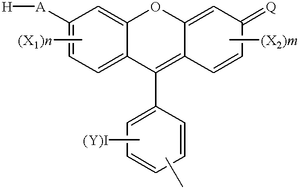
where
A represents O or NH,
Q represents O, S or NH,
X¹ represents hydrogen, halogen, nitro, hydroxyl, carboxyl (COOH), sulpho (SO₃H) or alkyl,
X² represents hydrogen, halogen, nitro, hydroxyl, carboxyl (COOH), sulpho (SO₃H) or alkyl,
Y represents halogen, alkyl, carboxyl (COOH) or sulpho (SO₃H),
l represents the indices 0 to 4, and
m, n represents the indices 0 to 3.

13. Substance according to claim 12, characterized in that
A represents O.

14. Substance according to claim 12 or 13, characterized in that
Q represents O.

15. Substance according to one of claims 12 to 14, characterized in that
X¹ represents hydrogen, fluorine, chlorine, bromine or alkyl having 1 to 4 carbon atoms, and
X² represents hydrogen, fluorine, chlorine, bromine or alkyl having 1 to 4 carbon atoms.

16. Substance according to one of claims 12 to 15, characterized in that
X¹ represents hydrogen, fluorine, chlorine, methyl or ethyl, and
X² represents hydrogen, fluorine, chlorine, methyl or ethyl.

17. Substance according to one of claims 11 to 16, characterized in that the hydrocarbon chain of the linker has up to 10 carbon atoms.

18. Substance according to one of claims 11 to 17, characterized in that the carbocyclic or heterocyclic group of the linker has up to 10 carbon atoms and, if appropriate, up to 5 nitrogen atoms and, if appropriate, 1 or 2 oxygen or sulphur atoms.

19. Substance according to one of claims 11 to 18, characterized in that the substance has the following structure:
Z¹—A¹—Z²—
where
A¹ represents a single bond, O or S,
Z¹ represents a carbocyclic or heterocyclic group having in each case up to 10 carbon atoms and, if appropriate, up to 5 nitrogen atoms and, if appropriate, 1 or 2 oxygen or sulphur atoms, and
Z² represents a carbocyclic or heterocyclic group having in each case up to 10 carbon atoms and, if appropriate, up to 5 nitrogen atoms and, if appropriate, 1 or 2 oxygen or sulphur atoms.

20. Substance according to claim 19, characterized in that
A¹ represents a single bond,
Z¹ represents one of the following groups 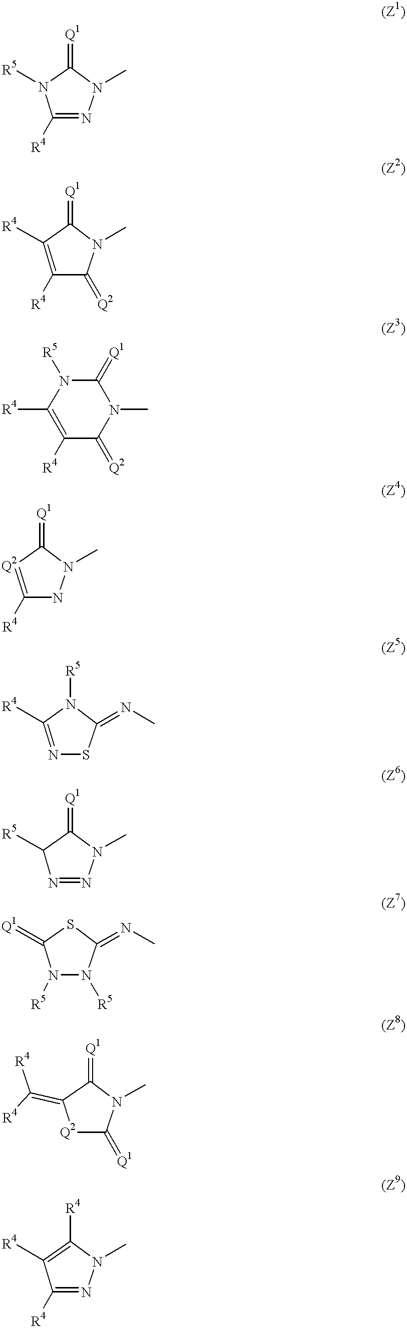
where
Q¹ represents O or S,
Q² represents O or S,
R⁴ represents hydrogen, amino, nitro, cyano, carboxyl, carbamnoyl, thio-carbamoyl, halogen, alkyl which has 1 to 6 carbon atoms and which is optionally substituted by cyano, halogen or C₁-C₄-alkoxy, or represents alkenyl or alkinyl, each of which has 2 to 6 carbon atoms and each of which is optionally substituted by halogen or represents alkoxy or alkoxycarbonyl, each of which has 1 to 6 carbon atoms in the alkyl groups and each of which is optionally substituted by cyano, halogen or C₁-C₄-alkoxy, or represents alkenyloxy or alkinyloxy, each of which has 3 to 6 carbon atoms and each of which is optionally substituted by halogen, or represents alkylthio which has 1 to 6 carbon atoms which is optionally substituted by cyano, halogen or C₁-C₄-alkoxy, or represents alkenylthio or alkinylthio, each of which has 3 to 6 carbon atoms and each of which is optionally substituted by halogen, or represents alkylamino or dialkylamino, each of which has 1 to 6 carbon atoms in the alkyl groups, or represents cycloalkyl or cycloalkylalkyl, each of which has 3 to 6 carbon atoms in the cycloalkyl groups and, if appropriate, 1 to 4 carbon atoms in the alkyl moiety and each of which is optionally substituted by cyano, halogen or C₁-C₄-alkyl,
R⁵ represents hydrogen, hydroxyl, amino, cyano, or represents alkyl, alkoxy, alkoxycarbonyl or alkylamino, each of which has up to 6 carbon atoms and each of which is optionally substituted by cyano, halogen or C₁-C₄-alkoxy, or represents alkenyl or alkinyl, each of which has up to 6 carbon atoms and each of which is optionally substituted by halogen, or represents cycloalkyl or cycloalkylalkyl, each of which has 3 to 6 carbon atoms in the cycloalkyl groups and, if appropriate 1 to 4 carbon atoms in the alkyl moiety and each of which is optionally substituted by cyano, halogen or C₁-C₄-alkyl, or represents phenyl or phenyl-C₁-C₁-C₁-C₁-C₁-C₁-C₄-alkyl each of which is optionally substituted by nitro, cyano, halogen, C₁-C₄-alkyl, C₁-C₄-halogeno-alkyl, C₁-C₄-alkoxy or C₁-C₄-halogenoalkoxy,
Y¹ represents O, S, SO, SO₂, NH, N(alkyl) or methylene and
Y² represents a single bond or O, S, SO, SO₂, NH or N(alkyl),
Y¹ and Y² being different in each individual case,
and
Z² represents the following group 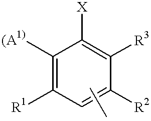
where
R¹ is hydrogen, nitro, cyano or halogen,
R² is nitro, hydroxyl, cyano, carbamoyl, thiocarbamoyl, or represents alkyl or alkoxy, each of which has 1 to 4 carbon atoms and each of which is optionally substituted by halogen, and
R³ represents nitro, hydroxyl, mercapto, amino, hydroxyamino, cyano, carboxyl, carbamoyl, thiocarbamoyl, halogen, or represents alkyl, alkylcarbonyl, alkoxy, alkoxycarbonyl, alkylthio, alkylsulphinyl, alkylsulphonyl or alkylamino, each of which has 1 to 6 carbon atoms in the alkyl groups and each of which is optionally substituted by cyano, carboxyl, carbamoyl, halogen, C₁-C₄-alkoxy or C₁-C₄-alkoxy-carbonyl, or represents alkylsulphonylamino, N,N-bis-alkylsulphonyl-amino, N-alkylcarbonyl-N-alkylsulphonyl-amino, each of which has 1 to 4 carbon atoms in the alkyl groups and each of which is optionally substituted by halogen, or represents alkenyl, alkenyloxy, alkenylthio, alkenylamino, alkinyl, alkinyloxy, alkinylthio, alkinylamino, each of which has up to 6 carbon atoms in the alkenyl or alkinyl groups and each of which is optionally substituted by cyano, carboxyl, carbamoyl, halogen or C₁-C₄-alkoxy-carbonyl, or represents cycloalkyl, cycloalkyloxy, cycloalkylthio, cycloalkylamino, cycloalkyl-sulphonylamino, cycloalkylalkyl, cycloalkylalkoxy, cyclo-alkylalkylthio or cycloalkylalkylamino, each of which has 3 to 6 carbon atoms in the cycloalkyl groups and, if appropriate, 1 to 4 carbon atoms in the alkyl moieties and each of which is optionally substituted by cyano, carboxyl, carbamoyl, halogen or C₁-C₄-alkyl, or represents aryl, aryloxy, arylthio, arylamino, arylalkyl, arylalkoxy, arylalkylthio, arylalkylamino, N-arylcarbonyl-N-alkylsulphonyl-amino, each of which has 6 or 10 carbon atoms in the aryl groups and, if appropriate, 1 to 4 carbon atoms in the alkyl moiety and each of which is optionally substituted by nitro, cyano, carboxyl, carbamoyl, halogen, C₁-C₄-alkyl, C₁-C₄-halogenoalkyl, C₁-C₄-alkoxy, C₁-C₄-halogenoalkoxy or C₁-C₄-alkoxy-carbonyl, and
X represents hydrogen or halogen.

21. Substance according to claim 19, characterized in that
A¹ represents O,
Z¹ represents one of the following groups 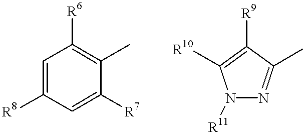
where
R⁶ represents cyano or halogen,
R⁷ represents hydrogen or halogen,
R⁸ represents cyano, halogen, or represents halogenoalkyl or halogenoalkoxy, each of which has 1 to 4 carbon atoms,
R⁹ represents hydrogen or halogen,
R¹⁰ represents halogenoalkyl or halogenoalkoxy, each of which has 1 to 4 carbon atoms, and
R¹¹ represents alkyl having 1 to 4 carbon atoms,
and
Z² represents the following group 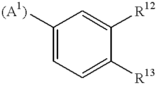
in which
R¹² represents carboxyl, or represents alkoxy or alkoxycarbonyl, each of which has up to 4 carbon atoms and each of which is optionally substituted by cyano, halogen or C₁-C₄-alkoxy, and
R¹³ represents cyano, carbamoyl, thiocarbamoyl, halogen or represents halogenoalkyl or halogenoalkoxy, each of which has 1 to 4 carbon atoms.

22. Substance according to claim 19, characterized in that the substance has the following general formula: 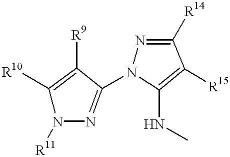
in which
R⁹, R¹⁰ and R¹¹ are as defined in claim 20,
R¹⁴ represents hydrogen, halogen or alkyl having 1 to 4 carbon atoms, and
R¹⁵ represents nitro, cyano, carbamoyl, thiocarbamoyl, or represents alkyl or alkoxy, each of which has 1 to 4 carbon atoms and each of which is optionally substituted by cyano, halogen or C₁-C₄-alkoxy.

23. Assay system comprising
a) containers with mixtures as defined in one of claims 1 to 10,
b) a device for irradiating the mixtures of plane-polarized light of a wavelength which excites the fluorescent substance to emit light, and
c) a device for measuring the fluorescence polarization values or the anisotropy values of the light emitted.

24. Use of a substance according to one of claims 11 to 22 for fluorescence correlation spectroscopy.

25. Substances which interact with the enzyme protoporphyrinogen oxidase and which have been identified by the method according to one of claims 1 to 10.
